(19) United States
(12) Patent Application Publication
(10) Pub. No.: US 2019/0330143 A1
(43) Pub. Date: Oct. 31, 2019

(54) PREPARATION METHOD FOR REMOVING TRIURET CAUSING TURBIDITY IN UREA WATER

(71) Applicant: Soultec Co., Ltd, Gyeongsangnam-do (KR)

(72) Inventor: Sang Won Shin, Seoul (KR)

(21) Appl. No.: 16/344,154

(22) PCT Filed: Sep. 5, 2017
(86) PCT No.: PCT/KR2017/009677

§ 371 Date: Apr. 23, 2019

(30) Foreign Application Priority Data
Oct. 25, 2016 (KR) 10-2016-0139118

(51) Int. Cl. C07C 273/16; C07C 275/02; B01F 13/08; B01D 61/14

(52) U.S. Cl. CPC C07C 273/16; B01D 61/142; B01F 13/08; C07C 275/02

(57) ABSTRACT

The present disclosure relates to a preparation method for removing triuret causing turbidity in a urea solution and to a urea solution prepared using the method, and more particularly, to a preparation method, in which urea is introduced to ultrapure water at 15° C. to 24° C. in a magnetic tank with a size of 1 m³ to 20 m³, and the mixture is stirred by using a magnetic mixer and filtered through a hollow-fiber type ultrafiltration membrane (having a pore size in a range of 0.01 μm to 0.3 μm) such that a 32.5% automotive urea solution has a turbidity of 0.02 NTU to 0.2 NTU, and the triuret causing turbidity in a urea solution and reducing conversion efficiency to NH₃ in selective catalytic reduction (SCR) is removed. To achieve the purpose of the present disclosure, there is provided a method of preparing a urea solution by dissolving urea in ultrapure water, wherein the 32.5% automotive urea solution is provided by a preparation method including the steps of: a step of preparing ultrapure water at 15° C. to 24° C.; a step of moving the ultrapure water into a magnetic mixer tank; a step of introducing urea to the ultrapure water contained in the magnetic mixer tank; a step of operating a magnetic mixer to induce vortex effect for mixing and stirring the ultrapure water with urea to prepare a urea solution; and a step of filtering by passing the urea solution including triuret through a hollow-fiber type ultrafiltration filter (U/F) to filtrate 20 L of triuret and impurities per 1,000 L of the urea solution, while maintaining a low temperature of the urea solution resulting from the stirring (S5).

### TECHNICAL FIELD

The present disclosure relates to a preparation method for removing triuret causing turbidity in a urea solution and to a urea solution prepared using the method, and more particularly, to a preparation method for removing triuret causing turbidity in a urea solution, in which urea is added to water of 15° C. to 24° C. for an automotive urea solution or water of 30° C. to 37° C. for marine and industrial urea solutions, and the mixture is stirred by using a magnetic mixer and filtered through a hollow-fiber type ultrafiltration membrane (having a pore size of 0.05 μm) such that a high-purity urea solution, which has a turbidity in a range of 0.02 NTU to 0.2 NTU and improved conversion efficiency to NH₃ by removing triuret in selective catalytic reduction (SCR), may be prepared.

### BACKGROUND ART

A urea solution is a liquid chemical material prepared by mixing urea that is a raw material for a urea fertilizer and water. Urea solutions include an automotive urea solution having a concentration of 31.8% to 33.2% and marine and industrial urea solutions having a concentration of 40%. The urea solution is used in purifying nitrogen oxides. Nitrogen oxides cause various respiratory diseases such as bronchitis and pneumonia and are known to be a major cause of optical smog and acid rain. Research has shown that more than twice the number of traffic accident deaths is attributed to automobile exhaust, which has a great influence on everyday lives. Accordingly, as a part of the reduction of greenhouse gas emissions, which has been challenged steadily for a long time, especially in developed countries, regulation of vehicle exhaust gas has become increasingly strict. Republic of Korea is following the European standards for ‘diesel emission regulation’.

The selective catalytic reduction (SCR) catalyst system may achieve 90% or higher DeNOₓ performance by using ammonia as a reducing agent. However, since ammonia is in a vapor phase, storing ammonia is difficult, and leakage of ammonia adversely affects human bodies. Thus, it is nearly impossible to use ammonia. The urea-SCR system may compensate for shortcomings of the SCR system using ammonia as a catalyst.

The urea-SCR is a technique of purifying nitrogen oxides using a urea solution. The urea-SCR is referred to as diesel exhaust fluid (DEF) in USA and as AdBlue in Europe. A freezing point of the urea solution may greatly vary depending on the temperature. A freezing point thereof with a concentration of 32.5% is −11° C., which is the lowest. Thus, when a standard is determined, a concentration of an automotive urea is set to be 32.5%, a concentration of a marine urea to be 40%, and a concentration of an industrial urea to be 40%.

When an engine of an automobile of the urea-SCR system is running, a urea solution is consistently used in an amount in a range of 4% to 6% relative to an amount of fuel. Research has shown that SCR improves fuel efficiency by an average of 3% to 5% over an EGR+DPF system in a system to cope with the strengthened exhaust emission regulation. Thus, using a urea solution may additionally cost, however, the total running expense of the urea-SCR system is economical due to the fuel efficiency effect.

In the urea-SCR system, when exhaust gas generated from a diesel engine is passing through SCR catalyst system, a urea solution may be injected thereinto through a urea solution injection apparatus to purify nitrogen oxides in the exhaust g as.

For dissolution of urea in preparation of a urea solution for use in the urea-SCR system in the related art, pure water was heated up to 40° C. for an automotive urea solution, and for industrial and marine urea solutions, heat is supplied during a dissolution process in addition to the method of heating pure water up to 40° C.

When urea is dissolved, an endothermic reaction may occur. Thus, as the reaction progresses, the water temperature may decrease, and thus the dissolution rate may also be greatly decreased. In dissolution using a general stirrer, the temperature is raised to prevent the dissolution rate from being decreased.

Considering that the average water temperature of the four seasons in Republic of Korea is 10.5° C., the method of heating pure water or water up to 40° C. to increase the dissolution rate of urea requires very high energy consumption.

In addition, to apply a heating method in the related art, additional equipment such as heating equipment is inevitably needed, and therefore, the size of an apparatus for preparing a urea solution is inevitably increased as a whole, resulting in low efficiency and low economic efficiency due to an increase in the preparing cost.

In addition, technology of preparing and refining a urea solution by using a water circulation system used overseas has a disadvantage in that urea is not properly dissolved, and it takes more than 30 minutes for dissolution, which is very inefficient in terms of productivity. Particularly, as the time required for dissolution increases, CO₂ in the atmosphere is continually injected thereto to generate a salt.

Therefore, in the technology of preparing and refining a urea solution, it is necessary to widespread an apparatus for preparing and purifying a urea solution of a high purity within a short time by using only an auxiliary function with or without a heating method.

Urea is mixed with ultrapure water in preparation of a urea solution. Here, triuret, which is an impurity of urea, melts in the urea solution because triuret has a melting point of 23.1° C., causing turbidity in a distribution process. Vehicles using the urea solution have a problem of failures due to accumulation of cyanuric acid in expensive SCR systems. In addition, a urea solution including triuret reduces conversion efficiency to NH₃ in SCR.

In KR 10-1640401 and KR 10-15445030, a method of mixing solid urea with pure water for forced circulation is suitable for preparation of a urea solution less than 5 tons, and it is difficult to filter 5 tons or more per hour by using the hollow-fiber ultrafiltration membrane filter and ion-exchange resin described therein.

That is, the technology described therein is not effective.

### DESCRIPTION OF EMBODIMENTS

### Technical Problem

The present disclosure is invented to resolve the aforementioned problems in the related art. Provided is a preparation method of a high-purity urea solution preparable at room temperature for an automotive urea solution in which conversion efficiency to NH₃ in SCR is improved by removing triuret without a chemical reaction due to impurities regardless of distribution in winter and summer and a temperature of a place of usage, thereby protecting SCR systems.

### Solution to Problem

According to an aspect of the present disclosure, a preparation method of a urea solution by dissolving urea in ultrapure water, in which an automobile urea solution is provided by a preparation method including the steps of:

a step of preparing ultrapure water at 15° C. to 24° C.;

a step of moving the ultrapure water into a magnetic mixer;

a step of introducing urea to the ultrapure water contained in the magnetic mixer;

a step of operating a magnetic mixer to induce vortex effect for mixing and stirring the ultrapure water with urea to prepare a urea solution; and

a step of filtering by passing the urea solution including triuret through a hollow-fiber type ultrafiltration filter (having a pore size in a range of 0.01 μm to 0.3 μm) while maintaining a low temperature of the urea solution resulting from the stirring (S5).

According to an aspect of the present disclosure, the preparation method may further include: a step of washing the hollow-fiber type ultrafiltration filter either in a forward direction or a reverse direction with hot water when a filtration capacity per hour is less than a given capacity (S6) in the step of filtering by passing the urea solution including triuret through a hollow-fiber type ultrafiltration filter (having a pore size in a range of 0.01 μm to 0.3 μm), while maintaining a low temperature of the urea water resulting from the stirring (S5).

According to an aspect of the present disclosure, in the preparation method, several hollow-fiber ultrafiltration filters are alternately used to change a flow from a hollow-fiber ultrafiltration filter with a decreasing filtration capacity, which is currently used, to another hollow-fiber ultrafiltration filter, and the hollow-fiber ultrafiltration filter with a decreasing filtration capacity is then washed such that preparation of the urea solution is not interrupted in the step of washing the hollow-fiber type ultrafiltration filter either in a forward direction or a reverse direction (S6).

### Advantageous Effects of Disclosure

The preparation method in which triuret and biuret that cause turbidity in a urea solution are removed is advantageous in the case of an automotive urea solution in that ultrapure water at room temperature range of 15° C. to 24° C. is used, and thus a preparation process is simple without a heating process, a preparation time is short, and a production cost is reduced. In addition, in the present disclosure, a magnetic mixer is used to induce vortex effect, while stirring at a low temperature without heating. Thus, a production cost is reduced, a time for stirring is shortened, and stirring efficiency is improved. In addition, in the present disclosure, a urea solution at a low temperature passes through a general filter to protect a hollow-fiber ultrafiltration filter and then through the hollow-fiber ultrafiltration filter. Thus, triuret that agglomerates at a low temperature may be effectively filtered, which is advantageous. In addition, in the present disclosure, since a hollow-fiber ultrafiltration filter (having a pore size in a range of 0.01 μm to 0.3 μm) is used in filtration, when a filtration capacity is decreased due to accumulated triuret in the filter, triuret may be easily removed by hot water, and the filter may be re-used.

Two or more hollow-fiber ultrafiltration filters according to the present disclosure may be alternately used for continuous production that enables washing without ceasing a preparation process.

When a urea solution is prepared using the preparation method according to the present disclosure, a high-purity urea solution having a turbidity in a range of 0.02 NTU to 0.2 NTU and a pH in a range of 9 to 11 is prepared. Accordingly, conversion efficiency to NH₃ in SCR is improved, the urea solution may be distributed regardless of season, the urea solution may be stored regardless of a temperature of the storage, and SCR systems are protected to thereby prevent replacement loss.

### BRIEF DESCRIPTION OF DRAWINGS

FIG. 1 is a flow chart illustrating a preparation method according to an embodiment of the present disclosure;

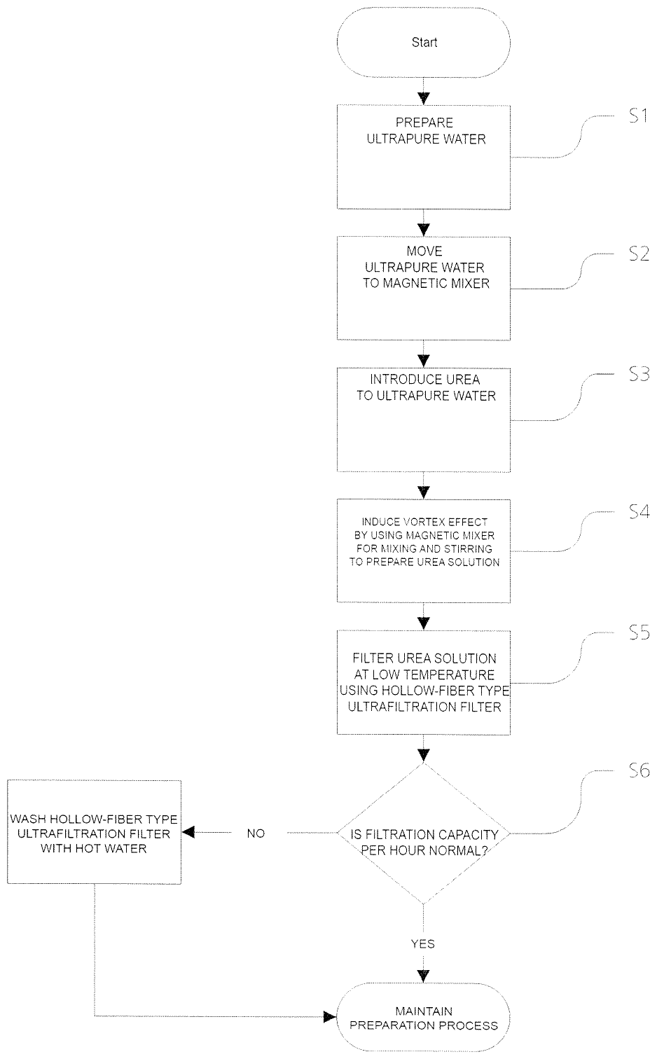

FIG. 2 is a front view of a magnetic mixer used in an embodiment of the present disclosure; and

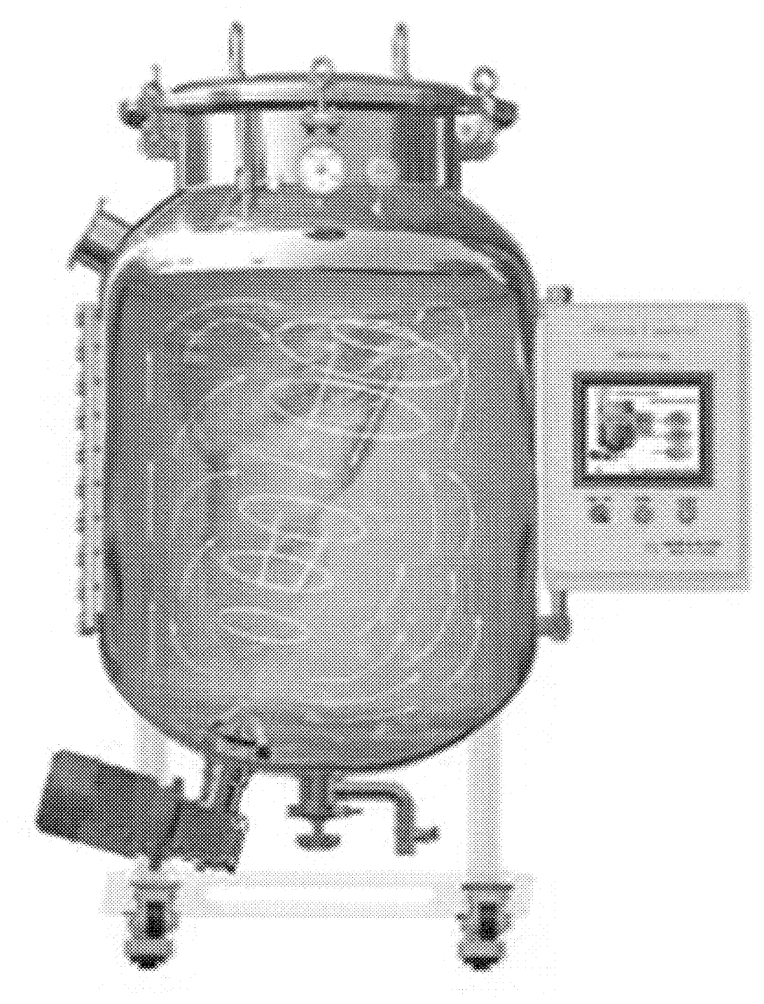

FIG. 3 is a perspective view of a hollow-fiber ultrafiltration filter in an embodiment of the present disclosure.

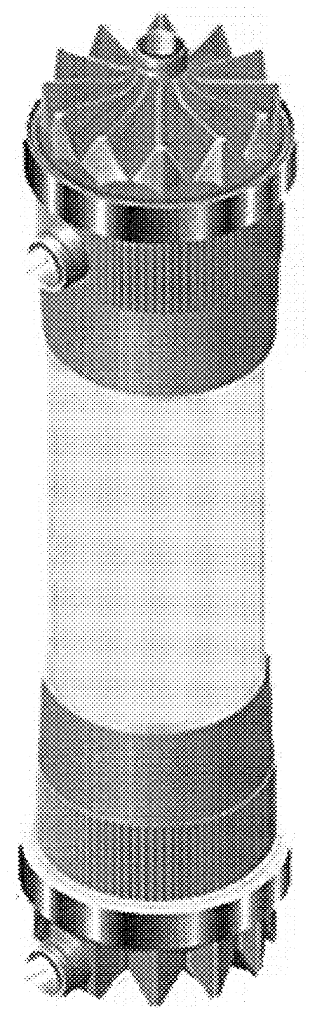

### BEST MODE

Hereinafter, preferred embodiments of the present disclosure will be described with reference to the accompanying drawings. In the following, the thicknesses of lines and the sizes of components shown in the drawings may be exaggerated for clarity and convenience of explanation. In addition, the terms described herein are defined in consideration of functions of the present disclosure, which may vary depending on the intention or custom of a user or an operator. Therefore, definitions of these terms should be understood based on the contents throughout the specification.

In addition, the following embodiments are not intended to limit the scope of the present disclosure, and these are illustrative purposes only. Various embodiments may be made within the spirit of the present disclosure.

A first step (S1) is preparing ultrapure water at 15° C. to 24° C. (S1) in the case of an automotive urea solution.

Ultrapure water may be prepared by various methods, however, in a preferred embodiment of the present disclosure, ultrapure water is obtained by mixing a carbon filter filtration system, a reverse osmosis filtration system, and an ion exchange filtration system.

A second step (S2) is moving the ultrapure water into a magnetic mixer tank.

A third step (S3) is introducing urea to the ultrapure water contained in the magnetic mixer tank.

A fourth step (S4) is operating a magnetic mixer for mixing and stirring the ultrapure water with urea.

Due to an endothermic reaction of urea, a stirring temperature may be dropped to −2° C. to −20° C. depending on an amount to be stirred.

When the temperature is dropped, mixing time may be excessively long when a propeller type mixer, i.e., a mixing method in the related art, is used. In this case, productivity may be poor, and dissolution may only be possible without ice formation by incubation at a temperature at −3° C.

However, the magnetic mixer applied to the present disclosure has a strong turning force and may effectively mix liquids that have strong viscosity even at the dropped stirring temperature without the incubation.

When the magnetic mixer is applied, vortex effect was generated in the mixing and stirring of the urea solution.

Accordingly, a 31.8% to 33.2% urea solution was produced within 10 minutes due to 360° strong rotation of the urea solution in a stirrer. In a preferred embodiment of the present disclosure, the time required to reach a required concentration for complete dissolution and the corresponding temperature change are shown in Experimental Example 1 in Table 1.

TABLE 1
Experimental Example 1

| Time (seconds) | Temperature (° C.) | Concentration (%) |
|---|---|---|
| Start | 18.8 | Urea injection |
| 90 | 11 | 15.8 |
| 150 | 9 | 17.9 |
| 210 | 6 | 23.5 |
| 270 | 3 | 27.7 |
| 330 | 2 | 31.5 |
| 390 | −2 | 32.5 |

When 743 liters (L) of ultrapure water is mixed with 358 kilograms (Kg) of urea at a water temperature of 19° C.

A fifth step (S5) is passing the urea solution (including triuret) through a hollow-fiber type ultrafiltration filter (having a pore size in a range of 0.01 μm to 0.3 μm) while maintaining a low temperature of the urea solution resulting from the stirring.

The urea solution prior to filtration has a turbidity in a range of 100 NTU to 300 NTU due to triuret having an enlarged grain size by association.

When the triuret not removed from the urea solution, the following problems may occur.

H₆N₄C₃O₃ (triuret)+250° C. (exhaust gas temperature)=(base) NH₃+(acid) H₃N₃C₃O₃ (cyanuric acid)

A decomposition point of cyanuric acid is in a range of 320° C. to 360° C. When a temperature of exhaust gas is 250° C., cyanuric acid is not decomposed and remains as a salt. Accordingly, cyanuric acid may remain in SCR, which may cause failure of SCR.

A natural conversion of a urea solution not including triuret to NH₃: 2 mol of NH₃ per 1 mol of the urea solution

CO(NH₂)₂→NH₃+HNCO

HNCO+H₂O→NH₃+CO₂

An unnatural conversion of a urea solution including triuret to NH₃ results in generation of 1 mol of NH₃ per 1 mol of the urea solution

When the urea solution is not completely decomposed due to inclusion of triuret, decomposition of the urea solution proceeds at downstream of a SCR catalyst, and the ammonia slip in which NH₃ is discharged is likely to occur.

A hollow-fiber ultrafiltration filter (having a pore size in a range of 0.01 μm to 0.3 μm) may be a general hollow-fiber ultrafiltration filter (U/F). A urea molecule (having a molecular weight of 60.06 g/mol) is smaller than a biuret molecule (C₂H₅N₃O₂, having a molecular weight of 103.081 g/mol) and triuret molecule (C₃H₆N₄O₃, having a molecular weight of 146.11 g/mol). In addition, triuret is agglomerated at a low temperature in the present disclosure, and thus, triuret molecules may easily be filtered by a filter. In a preferred embodiment of the present disclosure, by using a filter having a filter size similar to that of a triuret particle, triuret is filtered, and the filtration speed is fast. In a preferred embodiment of the present disclosure, by using several cylindrical hollow-fiber ultrafiltration filters having a height of 2,275 millimeters (mm) and a radius of 216 mm, 16,000 L per hour was filtered at maximum capacity.

A high-purity urea solution that underwent such filtration process has a turbidity in a range of 0.02 NTU to 0.2 NTU.

On the other hand, impurities, aldehydes, insoluble substances, and heavy metals that entered during a mixing process are also separated and removed in the filtration process.

Accordingly, improved conversion efficiency to NH₃ in SCR results in a high-purity urea solution.

A sixth step (S6) is washing the hollow-fiber type ultrafiltration filter (having a pore size in a range of 0.01 μm to 0.3 μm) either in a forward direction or a reverse direction with hot water when a filtration capacity per hour thereof is less than a given capacity.

As triuret is easily dissolved in hot water, triuret may be easily removed by such washing method.

Such washing method may be performed by automatic control. In an embodiment of the present disclosure, a flow is changed from a hollow-fiber ultrafiltration filter with a decreasing filtration capacity, which is currently used, to another hollow-fiber ultrafiltration filter, and the hollow-fiber ultrafiltration filter with a decreasing filtration capacity is then washed such that preparation of the urea solution is not interrupted.

A urea solution prepared as described above may be stably used without any chemical reaction due to impurities regardless of distribution in winter and summer and a temperature of a place of usage. For the above reasons, the turbidity is very low, and impurities are not visible to a naked eye. Also, conversion efficiency to ammonia gas is improved in SCR. In addition, since triuret is removed, expensive SCR systems such as a catalyst, an injector, and a urea solution filter are protected to thereby prevent replacement loss. In addition, since the automobile urea solution is not heated and is stirred at room temperature, boiler is not required to be used for heating. Therefore, energy cost loss per year may be prevented, resulting in an expected effect on greenhouse gas reduction and carbon emission.

1-3. (canceled)

4. A preparation method of a urea solution having a turbidity of 0.02 NTU or greater and 0.2 NTU or less and a pH in a range of 9 to 11 by more than 5 tons per hour by dissolving urea in ultrapure water, the preparation method being characterized by removing triuret that causes turbidity in the urea solution and reducing conversion efficiency to NH₃ in SCR, and the preparation method comprising:
a step of preparing ultrapure water at 15° C. to 24° C. (S1);
a step of moving the ultrapure water into a magnetic mixer tank having a size of 1 m³ to 20 m³ (S2);
a step of introducing urea to the ultrapure water contained in the magnetic mixer tank (S3);
a step of operating a magnetic mixer to induce vortex effect for mixing and stirring the ultrapure water with the urea to prepare a urea solution having a temperature reduced to −2° C. or lower and −20° C. or higher due to an endothermic reaction caused by the mixing (S4); and
a step of filtering the urea solution comprising triuret by passing the urea solution through a hollow-fiber type ultrafiltration filter (U/F) while maintaining a low temperature of the urea solution reduced by the stirring (S5).

5. The preparation method of claim 4, further comprising: a step of washing the hollow-fiber type ultrafiltration filter either in a forward direction or a reverse direction with hot water when a filtration capacity per hour is less than a given capacity (S6) in the step of filtering the urea solution comprising triuret by passing the urea solution through a hollow-fiber type ultrafiltration filter (having a pore size in a range of 0.01 μm to 0.3 μm), while maintaining a low temperature of the urea solution reduced by the stirring (S5).

6. The preparation method of claim 5, characterized by alternately using several hollow-fiber ultrafiltration filters to change a flow from a hollow-fiber ultrafiltration filter with a decreasing filtration capacity, which is currently used, to another hollow-fiber ultrafiltration filter and then wash the hollow-fiber ultrafiltration filter with a decreasing filtration capacity with hot water to allow preparation of the urea solution not to be interrupted in the step of washing the hollow-fiber type ultrafiltration filter (having a pore size in a range of 0.01 μm to 0.3 μm) either in a forward direction or a reverse direction (S6).
